(19) United States
(12) Patent Application Publication
(10) Pub. No.: US 2008/0286201 A1
(43) Pub. Date: Nov. 20, 2008

(54) Caspase-3 Substrate Comprising Imaging Agents

(76) Inventors: Benedicte Guilbert, Buckinghamshire (GB); Sue Champion, Buckinghamshire (GB); Alexander Mark Gibson, Buckinghamshire (GB); Sally-Anne Ricketts, Buckinghamshire (GB); Michelle Avory, Buckinghamshire (GB); Bente E. Arbo, Oslo (NO)

Correspondence Address: GE HEALTHCARE, INC., IP DEPARTMENT, 101 CARNEGIE CENTER, PRINCETON, NJ, 08540-6231, US

(21) Appl. No.: 11/815,360

(22) PCT Filed: Feb. 3, 2006
(86) PCT No.: PCT/GB2006/000398

§ 371 Date: May 19, 2008

(30) Foreign Application Priority Data
Feb. 4, 2005 (GB) 0502277.7

(51) Int. Cl. A61K 51/08; C12N 9/50

(52) U.S. Cl. 424/1.69; 435/219

(57) ABSTRACT

The present invention relates to diagnostic imaging agents for in vivo imaging. The imaging agents comprise a synthetic caspase-3 substrate peptide labelled with an imaging moiety suitable for diagnostic imaging in vivo. The invention also provides radiopharmaceutical compositions comprising the imaging agents, together with kits for the preparation of the radiopharmaceuticals. Also described are non-radioactive precursors suitable for the preparation of the imaging agents. The imaging agents are useful for the diagnostic imaging and or therapy monitoring in vivo of various disease states where caspase-3 is involved.

### FIELD OF THE INVENTION

The present invention relates to diagnostic imaging agents for in vivo imaging. The imaging agents comprise a synthetic caspase-3 substrate labelled with an imaging moiety suitable for diagnostic imaging in vivo.

### BACKGROUND TO THE INVENTION

Programmed cell death by apoptosis is a complex process, involving a large number of cellular processes with numerous levels of control. It is initiated by one of two pathways. The first is through an extrinsic pathway initiated via cell surface death receptors and the second is through intrinsic initiators, such as DNA damage by UV radiation. Both of these pathways culminate in the coordinated death of cells which requires energy and, unlike cell death by necrosis, does not involve an inflammatory response. Cells committed to apoptosis present ‘eat me’ signals on their cell surface, which invite other cells to consume them by phagocytosis.

Apoptosis is a critical event in numerous processes within the body. For example, embryonic development is totally reliant on apoptosis, and tissues that turnover rapidly require tight regulation to avoid serious pathological consequences. Failure to regulate apoptosis can give rise to cancers (insufficient cell death) and neuropathologies such as Alzheimer's disease (too much cell death). Furthermore, apoptosis can also be indicative of damaged tissues such as areas within the heart following ischaemia/reperfusion insults.

Annexin-5 is an endogenous human protein (RMM 36 kDa) which binds to the phosphatidylserine (PS) on the outer membrane of apoptotic cells with an affinity of around 10⁻⁹ M. ^{99m}Tc-labelled Annexin-5 has been used to image apoptosis in vivo [Blankenberg et al, J. Nucl. Med., 40, 184-191 (1999)]. There are, however, several problems with this approach. First, Annexin-5 can also enter necrotic cells to bind PS exposed on the inner leaflet of the cell membrane, which could lead to false-positive results. Second is the high blood pool activity, which is maintained for at least two hours after injection of labelled Annexin-5. This means that the optimal timing of imaging is between 10 and 15 h after injection [Reutelingsperger et al, J. Immunol. Meth., 265 (1-2), 123-32 (2002)], making it unsuitable for clinical decision making in patients with acute coronary syndromes. Furthermore, the clearance of annexin-5 occurs via the kidney and the liver, with a very strong background signal in the abdominal regions. This makes imaging of abdominal cell death (eg. in kidney transplants and tumour monitoring) impossible.

WO 99/67284 discloses chelator conjugates of cell membrane permeant peptides linked to a ‘functional linker moiety’ which confers target cell specificity for a diagnostic or pharmaceutically active substance. The diagnostic substance is chosen from: —a radionuclide, a relaxivity metal, a fluorochrome, a dye or an enzyme substrate. A wide range of permeant peptides are disclosed, including Tat peptides. The target cell specificity is preferably conferred by a peptide or protein binding motif, and many enzyme targets are described. Caspases, from caspase-1 to caspase-13, are mentioned as preferred protease-reactive sequences.

WO 01/89584 discloses at Examples 16 to 18 and 21 that a chelator conjugate of the caspase-3 substrate tetrapeptide DEVD (ie. Asp-Glu-Val-Asp) may be useful for in vivo imaging of apoptopic tissue using MRI or scintigraphy.

Haberkorn et al [Nucl. Med. Biol., 28, 793-798 (2001)] studied the pan-caspase inhibitor, Z-VAD-fink ie. benzyloxycarbonyl-Val-Ala-DL-Asp(O-methyl)-fluoromethylketone labelled with the radioisotope ¹³¹I as a potential apoptosis imaging agent. They found the absolute cellular uptake of the agent to be low, and attributed this to the trapping of only one inhibitor molecule per activated caspase. They concluded that a labelled caspase substrate should not suffer from this problem and would be a better approach for an imaging agent.

In an article published in June 2005, Bauer et al [J. Nucl. Med., 46(6), 1066-1074 (2005)] report on the uptake of ¹³¹I-radiolabelled caspase substrates which contain the DEVDG peptide sequence into apoptopic cells. The DEVDG peptides themselves are said to exhibit little cellular uptake, with conjugated Tat cell-penetrating peptides giving improved results. Bauer et al conclude that radiometal labels would be expected to further improve intracellular retention of the label within apoptopic cells by either the release of charged metal complexes or transcomplexation.

Caspase substrates have been reviewed by Fischer et al [Cell Death Diff., 10, 76-100 (2003)].

Radiopharmaceuticals for apoptosis imaging have been reviewed by Lahorte et al [Eur. J. Nucl. Med., 31, 887-919 (2004)].

There is therefore still a need for an apoptosis imaging agent which permits rapid imaging (eg. within one hour of injection), and with good clearance from blood and background organs.

### THE PRESENT INVENTION

Caspases are very specific proteases, showing an absolute requirement for cleavage after an aspartic acid moiety of a peptide [Thornberry et al., Science 281, 1312-16, (1998)]. The scissile amide bond is the amide bond linking the α-carboxy group of an aspartic acid residue (or “P1 residue”) to the next amino acid in the peptide sequence in the direction of the peptide C-terminus. The presence of at least four amino acids on the N-terminal side of the scissile amide bond is also necessary for efficient catalysis. The preferred tetrapeptide recognition motif differs significantly for the different caspases [Thornberry et al, J. Biol. Chem., 272(19), 17907-17911, (1997)].

At least fourteen different caspases have been identified in humans to date, which are designated caspase-1, caspase-2 etc. Caspases have been categorised into three main categories:
Group I caspases (eg. caspase-1, -4, and -5) which prefer the sequence WEHD;
Group II caspases (eg. caspase-2, -3, and -7), which prefer the sequence DExD;
Group III caspases (eg. caspase-6, -8 and -9) which prefer the sequence (L/V)ExD.

Based on mechanism of activation, caspases have been divided as follows:
initiators such as caspase-8 and -9;
effectors such as caspase-3, -6 and -7;
pro-inflammatory enzymes such as caspase-1, -4, -5, -11, -12 and -13.

For caspase-3, x in DExD can be A, P, L or V [Cohen et al, Biochem. J. 326, 1-16 1997)]—where conventional single letter amino acid abbreviations are used. It has now been found that synthetic caspase-3 substrates labelled with an imaging moiety are useful diagnostic imaging agents for in vivo imaging of those diseases of the mammalian body where abnormal apoptosis, especially where excessive apoptosis is involved. The imaging moiety is radioactive, and is a gamma-emitting radioactive halogen or a positron-emitting non-metal.

The present invention relates to substrates of caspase-3, which is also known as CPP32, and is a 29 kDa cysteine protease. The primary advantage of using a substrate approach is the possibility of signal amplification. Thus, radiolabelled caspase-3 substrates would be delivered into the apoptotic cell, which would then be cleaved by activated caspase-3 and the cleaved radiolabelled fraction retained within the apoptotic cell. Since activated caspase-3 can cleave multiple substrates during the apoptotic cascade in the cell, this approach could result in a significant amplification of the tracer signal, providing better target to background ratios.

### DETAILED DESCRIPTION OF THE INVENTION

In a first aspect, the present invention provides an imaging agent which comprises a labelled caspase-3 substrate of Formula I:

Z¹-(X¹)ₘ₁l-Asp(R¹)-Xaa1-Xaa2-Asp(R²)-(A)ₙ[IM] (I)
where:
Z¹ is attached to the N-terminus of X¹ or the Asp residue, and is H or a metabolism inhibiting group;

X¹ is a cell membrane permeable leader sequence peptide of 4 to 20 amino acids which facilitates cell membrane transport from the outside to the inside of a mammalian cell in vivo;
Xaa1 is Glu(R³) or Met;
Xaa2 is Val or is Gln when Xaa1 is Met;
Asp is aspartic acid;
-(A)ₙ- is a linker group wherein each A is independently —CR₂—, —CR═CR—, —C≡C—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group, an amino acid, a sugar or a monodisperse polyethyleneglycol (PEG) building block;
each R is independently chosen from H, C₁₋₄ alkyl, C₂₋₄ alkenyl, C₂₋₄ alkynyl, C₁₋₄ alkoxyalkyl or C₁₋₄ hydroxyalkyl;
R² and R³ are independently R′ groups which are attached at the carboxy side chain of the Asp or Glu amino acid residue, where each R′ is chosen from H, C₁₋₈ alkyl, C₂₋₈ alkoxyalkyl, C₅₋₁₂ aryl or C₅₋₁₆ aralkyl;
m₁ is 0 or 1;
n is an integer of value 0 to 10;
IM is an imaging moiety which comprises a gamma-emitting radioactive halogen or a positron-emitting radioactive non-metal, wherein following administration of said labelled caspase-3 substrate to the mammalian body in vivo, the imaging moiety can be detected externally in a non-invasive manner.

The Asp(R¹)-Xaa1-Xaa2-Asp(R²) is a caspase-3 tetrapeptide substrate motif, hence the imaging agents of the present invention comprise synthetic caspase-3 substrates labelled with an imaging moiety.

Preferred imaging agents do not undergo facile metabolism in vivo, and hence most preferably exhibit a half-life in vivo of 60 to 240 mins in humans. The imaging agent is preferably excreted via the kidney (ie. exhibits urinary excretion). The imaging agent preferably exhibits a signal-to-background ratio at apoptotic foci of at least 1.5, most preferably at least 5, with at least 10 being especially preferred. Clearance of one half of the peak level of imaging agent which is either non-specifically bound or free in vivo, preferably occurs over a time period less than or equal to the radioactive decay half-life of the radioisotope of the imaging moiety.

The molecular weight of the imaging agent is suitably up to 5000 Daltons. Preferably, the molecular weight is in the range 150 to 3000 Daltons, most preferably 200 to 1500 Daltons, with 300 to 800 Daltons being especially preferred.

Caspase-3 can be expressed in almost all tissues at high levels relative to other caspases, and exhibits high catalytic activity compared to other Group II caspases. Caspase-3 is, however, only expressed in active form during apoptosis. This forms the basis for the labelled substrates of the present invention being viable imaging agents for apoptopic diseases, with good signal-to-noise.

Since the caspases are intracellular proteases, the imaging agents of the present invention exhibit good cell membrane permeability. This can be achieved via two approaches or combinations thereof. Firstly, peptides with acidic groups (such as carboxylic acid functions) will have increased cell permeability when they are present as esters rather than as free acids. These esters correspond to the R¹, R² and R³ groups of Formula I, where R′ is not H. Furthermore, the degree of cell permeability vs the degree of lipophilicity causing unfavourable pharmacokinetics, can be fine tuned by varying the nature of the esters used. The presence of different esterases within the cell will liberate the desired free acid form once the imaging agent has crossed the cell membrane. Hence, in a preferred embodiment, at least one of R¹, R² and R³ is C₁₋₈ alkyl. Preferably two or more of the R¹, R² and R³ groups are C₁₋₈ alkyl. When R¹, R² or R³ is C₁₋₈ alkyl, preferred alkyl groups are methyl, cyclohexyl and heptyl, most preferably methyl and cyclohexyl.

Secondly, to facilitate cell membrane transport, the imaging agents of the present invention preferably comprise a “leader sequence” (X¹) as defined below, ie. m₁ is preferably 1. The leader sequence is attached at the N-terminus of the caspase-3 substrate peptide. The “leader sequence” (X¹) group of the present invention is a 4- to 20-mer amino acid peptide which facilitates cell membrane transport. This is important since caspase-3 is an intracellular enzyme, and hence the imaging agents must be capable of crossing cell membranes. The leader sequence is thus useful to transport the imaging agent into the apoptotic cell, and also to transport the uncleaved peptide out of normal (ie. non-apoptotic cells). When one or more of R¹, R² or R³ is C₁₋₈ alkyl, the leader sequence is still useful to allow the imaging agent to exit cells that do not contain activated caspase-3, but inside which non-specific esterases could still remove the ester groups. Once the esters are hydrolysed, the uncleaved imaging agent might not be sufficiently lipophilic to exit cells which do not contain caspase-3, and this would lead to non-specific uptake. The latter roles help to improve the selective target-to-background of the imaging agent at the desired imaging site in vivo, and show why a leader sequence is preferred.

By the term “amino acid” is meant an L- or D-amino acid, amino acid analogue (eg. napthylalanine) or amino acid mimetic which may be naturally occurring or of purely synthetic origin, and may be optically pure, i.e. a single enantiomer and hence chiral, or a mixture of enantiomers. Conventional 3-letter or single letter abbreviations for amino acids are used herein. Preferably the amino acids of the present invention are optically pure. Suitable leader sequence peptides are known in the art, and include: tachyplesin derivatives; protegrin derivatives; cell membrane-permeable motifs such as a poly-Arg sequence; a β-peptide like β-(Val-Arg-Arg)ₙ or permeation motifs of viral proteins can be used, eg motifs based on the HIV-1 Tat protein basic peptide [Fawell et al, PNAS, 91: 664-68 (1994)]. β-peptides consist of β-amino acid residues (ie. having one extra —CH₂— in the backbone) as opposed to α-amino acids, and are more stable towards proteolytic degradation and form well-defined secondary structures [see T. B. Potocky et al, J Biol Chem., 278(50), 50188-94 (2003) and references cited therein]. Specific “leader sequences” and references thereto are given in Table 1 below:

TABLE 1
Leader sequences.

| | Leader Sequence | Description | Ref |
|---|---|---|---|
| 1 | CNSRLHLR and | Vascular targeting | Pasqualini J. Nucl. Med., |
| | CENWWGDV | with phage peptide | 43(2): 159-62 (1999). |
| | | libraries | |
| 2 | KWSFRVSYRGISYRRSR | Tachyplesin derivative | WO 99/07728; |
| | | | WO 00/32236; Nakamura |
| | | | et al J Biol Chem. 15; |
| | | | 263(32): 16709-13 (1988).; |
| | | | Tamura et al Chem. Pharm. |
| | | | Bull. Tokyo 41, 978-980 |
| | | | (1993). |
| 3 | AWSFRVSYRGISYRRSR | Tachyplesin derivative | WO 99/07728 |
| 4 | RKKRRQRRR | HIV-1 Tat₄₉₋₅₇ | Mie et al Biochem Biophys |
| | | | Res Commun. 24; 310(3): |
| | | | 730-4 (2003); Potocky et al |
| | | | J. Biol Chem. 278(5), |
| | | | 50188-50194 (2003). |
| 5 | RRLSYSRRRF | Protegrin derivative | WO 99/07728. |
| 6 | RGGRLSYSRRRFSVSVGR | Protegrin | WO 00/32236; Kokryakov |
| | | | et al FEBS Lett.; |
| | | | 327(2): 231-6 (1993). |
| 7 | RGGRLSYSRRRFSTSTGR | Tropic protegrin | WO 99/07728; |
| | | (SynB1) | WO 00/32236. |
| 8 | PRPRPLPFPRPGPPGPRPIPR | Ip (Bac7) | |
| 9 | RQIKIWFQNRRMKWKK | Penetratin | |
| 10 | RGGGLSYSRRRFSTSTGR | Tropic protegrin | |
| | | analogue | |
| 11 | ILPWKWPWWPWRR | Ip (Indolicin) | |
| 12 | FKCRRWQWRMKKLGA | Ip (Lferrin B) | |
| 13 | RLSRIVVIRVSR | Ip (Dodecapeptide) | |

The “leader sequence” does not provide biological targeting in vivo, but it can help to provide more rapid clearance from background organs in vivo. Thus, eg. ^{99m}Tc-labelled Tat peptides have been shown to exhibit more rapid renal clearance in vivo than other radiolabelled peptides [Polyakov et al, Bioconj. Chem., 11, 762-771 (2000)].

Preferred leader sequences peptides are Tat peptides, tachyplesin derivatives and protegrin derivatives. Especially preferred leader sequences are described by Gammon et al, [Bioconj. Chem., 14, 368-376 (2003)], and include RKKRR-Orn-RRR, RRRRRRRPR and β-(VRR)₄,where Orn is ornithine.

By the term “metabolism inhibiting group” (Z¹) is meant a biocompatible group which inhibits or suppresses in vivo metabolism of the peptide or amino acid at the amino terminus. Such groups are well known to those skilled in the art and are suitably chosen from, for the peptide amine terminus: acetyl, Boc (where Boc is tert-butyloxycarbonyl), Fmoc (where Fmoc is fluorenylmethoxycarbonyl), benzyloxycarbonyl, trifluoroacetyl, allyloxycarbonyl, Dde [i.e. 1-(4,4-dimethyl-2,6-dioxocyclohexylidene)ethyl] or Npys (i.e. 3-nitro-2-pyridine sulfenyl). More lipophilic Z¹ groups have the advantage that, even if the ester groups have been cleaved by intracellular esterases of non-apoptopic cells in vivo, the imaging agent is still sufficiently lipophilic to cross the cell membrane. This is useful to minimise undesirable non-specific uptake in non-apoptopic cells. Hence, preferred metabolism inhibiting groups for the peptide N-terminus are acetyl (when m₁=1) and benzyloxycarbonyl (when m₁=0).

The term “labelled with” means that either a functional group comprises the imaging moiety, or the imaging moiety is attached as an additional species. When a functional group comprises the imaging moiety, this means that the ‘imaging moiety’ forms part of the chemical structure, and is a radioactive isotope present at a level significantly above the natural abundance level of said isotope. Such elevated or enriched levels of isotope are suitably at least 5 times, preferably at least 10 times, most preferably at least 20 times; and ideally either at least 50 times the natural abundance level of the isotope in question, or present at a level where the level of enrichment of the isotope in question is 90 to 100%. Examples of such functional groups include CH₃ groups with elevated levels of ¹¹C, and fluoroalkyl groups with elevated levels of ¹⁸F, such that the imaging moiety is the isotopically labelled ¹¹C or ¹⁸F atom within the chemical structure. The radioisotopes ³H and ¹⁴C are not suitable imaging moieties.

When the imaging moiety is a gamma-emitting radioactive halogen, the radiohalogen is suitably chosen from ¹²³I, ¹³¹I or ⁷⁷Br. A preferred gamma-emitting radioactive halogen is ¹²³I. When the imaging moiety is a positron-emitting radioactive non-metal, the imaging agent would be suitable for Positron Emission Tomography (PET). Suitable such positron emitters include: ¹¹C, ¹³N, ¹⁷F, ¹⁸F, ⁷⁵Br, ⁷⁶Br or ¹²⁴i. Preferred positron-emitting radioactive non-metals are ¹¹C, ¹³N, ¹²⁴I and ¹⁸F, especially ¹¹C and ¹⁸F, most especially ¹⁸F.

The imaging moiety is preferably a positron-emitting radioactive non-metal. The use of a PET imaging moiety has certain technical advantages, including:
(i) the development of PET/CT cameras allowing easy co-registration of functional (PET) and anatomical (CT) images for improved diagnostic information;
(ii) the facility to quantify PET images to allow accurate assessment for staging and therapy monitoring;
(iii) increased sensitivity to allow visualisation of smaller target tissues.

It is envisaged that one of the roles of the linker group -(A)ₙ- of Formula I is to distance IM from the active site of the caspase-3 substrate. This is particularly important when the imaging moiety is relatively bulky (eg. a radioiodine atom), so that interaction with the enzyme is not impaired. This can be achieved by a combination of flexibility (eg. simple alkyl chains), so that the bulky group has the freedom to position itself away from the active site and/or rigidity such as a cycloalkyl or aryl spacer which orientate the IM away from the active site. The nature of the linker group can also be used to modify the biodistribution of the imaging agent. Thus, eg. the introduction of ether groups in the linker will help to minimise plasma protein binding. When -(A)ₙ- comprises a polyethyleneglycol (PEG) building block or a peptide chain of 1 to 10 amino acid residues, the linker group may function to modify the pharmacokinetics and blood clearance rates of the imaging agent in vivo. Such “biomodifier” linker groups may accelerate the clearance of the imaging agent from background tissue, such as muscle or liver, and/or from the blood, thus giving a better diagnostic image due to less background interference. A biomodifier linker group may also be used to favour a particular route of excretion, eg. via the kidneys as opposed to via the liver.

By the term “sugar” is meant a mono-, di- or tri-saccharide. Suitable sugars include: glucose, galactose, maltose, mannose, and lactose. Optionally, the sugar may be functionalised to permit facile coupling to amino acids. Thus, eg. a glucosamine derivative of an amino acid can be conjugated to other amino acids via peptide bonds. The glucosamine derivative of asparagine (commercially available from Novabiochem) is one example of this:

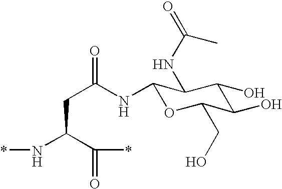

When -(A)ₙ- comprises a peptide chain of 1 to 10 amino acid residues, the amino acid residues are preferably chosen from glycine, lysine, arginine, aspartic acid, glutamic acid or serine. When -(A)ₙ- comprises a PEG moiety, it preferably comprises units derived from oligomerisation of the monodisperse PEG-like structures of Formulae IA or IB:

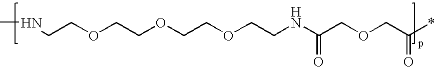

### 17-amino-5-oxo-6-aza-3,9,12,15-tetraoxaheptadecanoic acid of Formula IA (IA)

wherein p is an integer from 1 to 10 and where the C-terminal unit (*) is connected to the imaging moiety. Alternatively, a PEG-like structure based on a propionic acid derivative of Formula IB can be used:

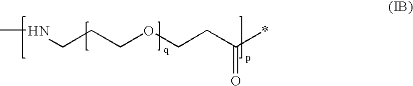

where p is as defined for Formula IA and q is an integer from 3 to 15.

In Formula IB, p is preferably 1 or 2, and q is preferably 5 to 12.

When the linker group does not comprise PEG or a peptide chain, preferred -(A)ₙ- groups have a backbone chain of linked atoms which make up the -(A)ₙ- moiety of 2 to 10 atoms, most preferably 2 to 5 atoms, with 2 or 3 atoms being especially preferred. A minimum linker group backbone chain of 2 atoms confers the advantage that the imaging moiety is well-separated so that any undesirable interaction is minimised.

Non-peptide linker groups such as alkylene groups or arylene groups have the advantage that there are no significant hydrogen bonding interactions with the conjugated caspase substrate, so that the linker does not wrap round onto the substrate. Preferred alkylene spacer groups are —(CH₂)_{d}— where d is 2 to 5. Preferred arylene spacers are of formula:

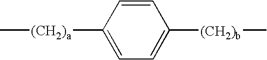

where: a and b are independently 0, 1 or 2.

The linker group -(A)ₙ- preferably comprises a diglycolic acid moiety, a glutaric acid, succinic acid, a polyethyleneglycol based unit or a PEG-like unit of Formula IA or IB.

Linker groups of the present invention preferably comprise a peptide chain of 1 to 10 amino acid residues, the amino acid residues are preferably chosen from glycine, lysine, arginine, aspartic acid, glutamic acid or serine. Preferred such amino acids are glycine and lysine.

The present invention requires the imaging moiety [IM] to be attached at a specific position. That is chosen because the Asp residue in the P1 position is important for substrate recognition and selectivity for caspases in general, and the four amino acids Asp-Glu-Val-Asp (DEVD) and Asp-Met-Gln-Asp (DMQD) have been identified as specific recognition motifs for Caspase-3. Modification at the carboxylic acid side chain of those aspartyl residues to attach an imaging moiety is therefore not preferred if substrate activity is to be preserved. Also, the imaging moiety is suitably located on the C-terminal side of the caspase-3 scissile amide bond, as described above. After cleavage by caspase-3, the fragment of the imaging agent containing the IM would possess an overall positive charge at physiological pH, and hence would be trapped within the apoptotic cell, since it will be too hydrophilic to cross cell membranes. This gives an enhanced imaging signal or signal-to-background ratio, due to specific enzymatic action. The positive charge is also expected to promote the association of the imaging moiety with intracellular proteins, most of which would be negatively charged. The feature is expected to be enhanced when the imaging agent (released after caspase cleavage) is chosen to include a linker group (A), wherein the amino acids of (A), are substituted with groups that would be protonated at physiological pH.

Certain caspase-3 fluorogenic and chromogenic substrates are commercially available, such as Z-DEVD-[Rhodamine110] (Cambridge Biosciences) and Ac-DEVD-[p-nitroaniline] (Calbiochem). Peptide-containing caspase-3 substrates and leader sequences of the present invention may also be obtained by conventional solid phase synthesis, as described in P. Lloyd-Williams, F. Albericio and E. Girald; Chemical Approaches to the Synthesis of Peptides and Proteins, CRC Press, 1997. The imaging agents of the present invention are suitably prepared by reaction with a precursor, as described in the second embodiment below.

In a second aspect, the present invention provides a precursor suitable for the preparation of the imaging agent of the first embodiment, which comprises a compound of Formula II:

Z¹-(X¹)ₘ₁-Asp(R¹)-Xaa1-Xaa2-Asp(R²)-(A)ₙ-[Y¹] (II)

where Z¹, X¹, m₁, R¹, Xaa1, Xaa2, Asp, R², A and n are as defined above, and Y¹ is a non-radioactive group which comprises a functional group or substituent capable of reaction with a source of the positron-emitting radioactive non-metal or gamma-emitting radioactive halogen to give the imaging agent of Formula (I). Preferred embodiments of Z¹, X¹, m₁, R¹, Xaa1, Xaa2, Asp, R², A and n are as described for the first aspect above.

The “precursor” suitably comprises a non-radioactive derivative of the caspase-3 substrate, which is designed so that chemical reaction with a convenient chemical form of the desired non-metallic radioisotope can be conducted in the minimum number of steps (ideally a single step), and without the need for significant purification (ideally no further purification) to give the desired radioactive product. Such precursors are synthetic and can conveniently be obtained in good chemical purity. The “precursor” may optionally comprise a protecting group (P^{GP}) for certain functional groups of the caspase-3 substrate. Suitable precursors are described by Bolton, J. Lab. Comp. Radiopharm., 45, 485-528 (2002).

By the term “protecting group” (P^{GP}) is meant a group which inhibits or suppresses undesirable chemical reactions, but which is designed to be sufficiently reactive that it may be cleaved from the functional group in question under mild enough conditions that do not modify the rest of the molecule. After deprotection the desired product is obtained. Protecting groups are well known to those skilled in the art and are suitably chosen from, for amine groups: Boc (where Boc is tert-butyloxycarbonyl), Fmoc (where Fmoc is fluorenylmethoxycarbonyl), trifluoroacetyl, allyloxycarbonyl, Dde [i.e. 1-(4,4-dimethyl-2,6-dioxocyclohexylidene)ethyl] or Npys (i.e. 3-nitro-2-pyridine sulfenyl); and for carboxyl groups: methyl ester, tert-butyl ester or benzyl ester. For hydroxyl groups, suitable protecting groups are: methyl, ethyl or tert-butyl; alkoxymethyl or alkoxyethyl; benzyl; acetyl; benzoyl; trityl (Trt) or trialkylsilyl such as tert-butyldimethylsilyl. For thiol groups, suitable protecting groups are: trityl and 4-methoxybenzyl. The use of further protecting groups are described in ‘Protective Groups in Organic Synthesis’, Theorodora W. Greene and Peter G. M. Wuts, (Third Edition, John Wiley & Sons, 1999).

Preferred precursors are those wherein Y¹ comprises a derivative which either undergoes direct electrophilic or nucleophilic halogenation; undergoes facile alkylation with a labelled alkylating agent chosen from an alkyl or fluoroalkyl halide, tosylate, triflate (ie. trifluoromethanesulphonate), mesylate, maleimide or a labelled N-haloacetyl moiety; alkylates thiol moieties to form thioether linkages; or undergoes condensation with a labelled active ester, aldehyde or ketone. Examples of the first category are:
(a) organometallic derivatives such as a trialkylstannane (eg. trimethylstannyl or tributylstannyl), or a trialkylsilane (eg. trimethylsilyl);
(b) a non-radioactive alkyl iodide or alkyl bromide for halogen exchange and alkyl tosylate, mesylate or triflate for nucleophilic halogenation;
(c) aromatic rings activated towards electrophilic halogenation (eg. phenols) and aromatic rings activated towards nucleophilic halogenation (eg. aryl iodonium, aryl diazonium, aryl trialkylammonium salts or nitroaryl derivatives).

Preferred derivatives which undergo facile alkylation are alcohols, phenols, amine or thiol groups, especially thiols and sterically-unhindered primary or secondary amines. Preferred derivatives which alkylate thiol-containing radioisotope reactants are maleimide derivatives or N-haloacetyl groups. Preferred examples of the later are N-chloroacetyl and N-bromoacetyl derivatives.

Preferred derivatives which undergo condensation with a labelled active ester moiety are amines, especially sterically-unhindered primary or secondary amines.

Preferred derivatives which undergo condensation with a labelled aldehyde or ketone are aminooxy and hydrazides groups, especially aminooxy derivatives.

The “precursor” may optionally be supplied covalently attached to a solid support matrix. In that way, the desired imaging agent product forms in solution, whereas starting materials and impurities remain bound to the solid phase. Precursors for solid phase electrophilic fluorination with ¹⁸F-fluoride are described in WO 03/002489. Precursors for solid phase nucleophilic fluorination with ¹⁸F-fluoride are described in WO 03/002157. The kit may therefore contain a cartridge which can be plugged into a suitably adapted automated synthesizer. The cartridge may contain, apart from the solid support- bound precursor, a column to remove unwanted fluoride ion, and an appropriate vessel connected so as to allow the reaction mixture to be evaporated and allow the product to be formulated as required. The reagents and solvents and other consumables required for the synthesis may also be included together with a compact disc carrying the software which allows the synthesiser to be operated in a way so as to meet the customer requirements for radioactive concentration, volumes, time of delivery etc. Conveniently, all components of the kit are disposable to minimise the possibility of contamination between runs and will be sterile and quality assured.

When the imaging moiety comprises a radioactive halogen, such as iodine, Y¹ suitably comprises: a non-radioactive precursor halogen atom such as an aryl iodide or bromide (to permit radioiodine exchange); an activated precursor aryl ring (e.g. phenol or aniline groups); an imidazole ring; an indole ring; an organometallic precursor compound (eg. trialkyltin or trialkylsilyl); or an organic precursor such as triazenes or a good leaving group for nucleophilic substitution such as an iodonium salt. Methods of introducing radioactive halogens (including ¹²³I and ¹⁸F) are described by Bolton [J. Lab. Comp. Radiopharm., 45, 485-528 (2002)]. Examples of suitable precursor aryl groups to which radioactive halogens, especially iodine can be attached are given below:

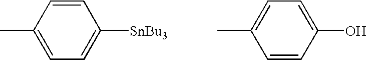

Both contain substituents which permit facile radioiodine substitution onto the aromatic ring. Alternative substituents containing radioactive iodine can be synthesised by direct iodination via radiohalogen exchange, e.g.

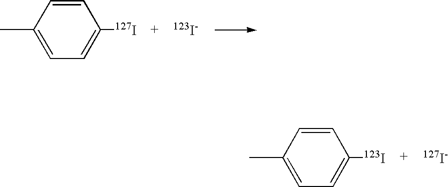

When the imaging moiety comprises a radioactive isotope of iodine the radioiodine atom is preferably attached via a direct covalent bond to an aromatic ring such as a benzene ring, or a vinyl group since it is known that iodine atoms bound to saturated aliphatic systems are prone to in vivo metabolism and hence loss of the radioiodine. An iodine atom bound to an activated aryl ring like phenol has also, under certain circumstances, been observed to have limited in vivo stability.

When the imaging moiety comprises a radioactive halogen, such as ¹²³I and ¹⁸F, Y¹ preferably comprises a functional group that will react selectively with a radiolabelled synthon and thus upon conjugation gives the imaging agent of Formula (I). By the term “radiolabelled synthon” is meant a small, synthetic organic molecule which is:
(i) already radiolabelled such that the radiolabel is bound to the synthon in a stable manner;
(ii) comprises a functional group designed to react selectively and specifically with a corresponding functional group which is part of the desired compound to be radiolabelled. This approach gives better opportunities to generate imaging agents with improved in vivo stability of the radiolabel relative to imaging agents with improved in vivo stability of the radiolabel relative to direct radiolabelling approaches.

A synthon approach also allows greater flexibility in the conditions used for the introduction of the imaging moiety. This is important when one or more of the R¹ to R³ groups of Formula (I) are C₁₋₈ alkyl, since in these cases the caspase-3 substrates of the present invention exhibit significant instability under basic conditions. In addition, they are therefore not suitable for conventional direct labelling approaches via nucleophilic displacement reactions under basic conditions.

Examples of precursors suitable for the generation of imaging agents of the present invention are those where Y¹ of Formula (II) comprises an aminooxy group, a thiol group, an amine group, a maleimide group or an N-haloacetyl group. A preferred method for selective labelling is to employ aminooxy derivatives of peptides as precursors, as taught by Poethko et al [J. Nuc. Med., 45, 892-902 (2004)]. Such precursors are then condensed with a radiohalogenated-benzaldehyde synthon under acidic conditions (eg. pH 2 to 4), to give the desired radiohalogenated imaging agent via a stable oxime ether linkage. Y¹ therefore preferably comprises an aminooxy group of formula —NH(C═O)CH₂—O—NH₂. Another preferred method of labelling is when Y¹ comprises a thiol group which is alkylated with radiohalogenated maleimide-containing synthon under neutral conditions (pH 6.5-7.5) eg. as taught by Toyokuni et al [Bioconj. Chem. 1, 1253-1259 (2003)] to label thiol-containing peptide substrates.

An additional preferred method of labelling is when Y¹ comprises an amine group which is condensed with the synthon N-succinimidyl 4-[¹²³]iodobenzoate at pH 7.5-8.5 to give amide bond linked products. The use of N-hydroxysuccinimide ester to label peptides is taught by Vaidyanathan et al [Nucl. Med. Biol., 19(3), 275-281 (1992)] and Johnstrom et al [Clin. Sci., 103 (Suppl. 48), 45-85 (2002)].

When R¹-R³ of Formula (I) are C₁₋₈ alkyl the especially preferred method for labelling the imaging agent precursor with a radiohalogen is when Y¹ comprises an aminooxy group.

Deiodination of mono-iodotyrosine (and to a greater extent di-iodotyrosine) was observed with some compounds in vivo. Use of D-tyrosine derivatives is expected to be one way of overcoming this problem. Alternative ways of incorporating radioiodine which are believed to overcome this in vivo deiodination problem are given in Table 2:

TABLE 2
Precursors for iodination and the corresponding iodinated products

| R^{a} | R^{b} |
|---|---|
| 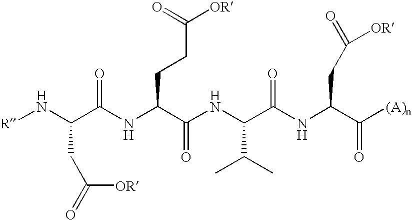 | 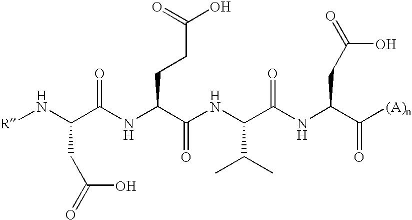 |
| Precursor | Product |
| 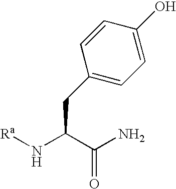 | 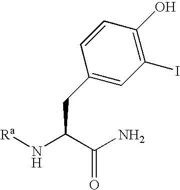 |
| 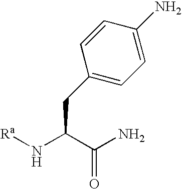 | 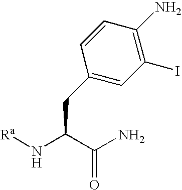 |
| 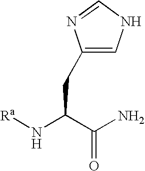 | 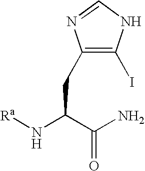 |
| 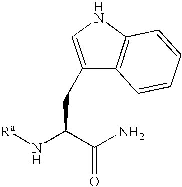 | 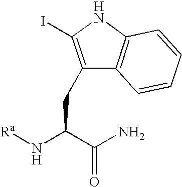 |
| 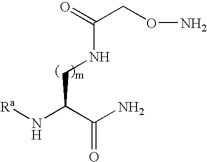 | 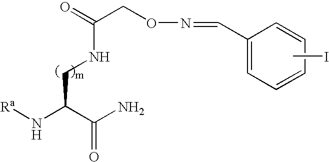 |
| 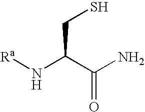 | 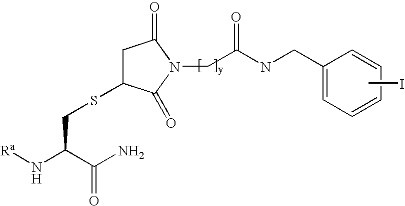 |
| 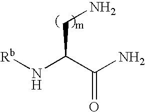 | 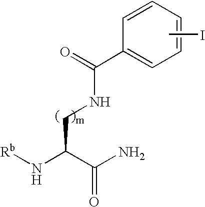 |
| 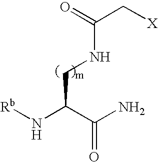 | 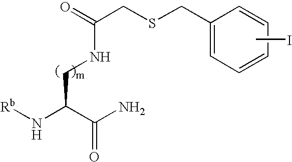 |
| 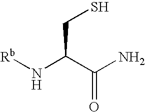 | 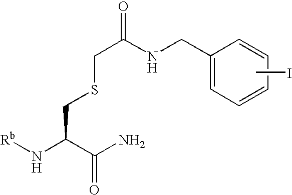 |

R′ is chosen from H, C₁₋₈ alkyl, C₂₋₈ alkoxyalkyl, C₅₋₁₂ aryl or C₅₋₁₆ aralkyl;
R″ = Z¹—(X¹)ₘ₁—
n = 1-10 and m = 1-4
y = C₁₋₁₀ alkyl, alkylaryl
X = Cl or Br.

When the imaging moiety comprises a radioactive isotope of fluorine the radiofluorine atom may form part of a fluoroalkyl or fluoroalkoxy group, since alkyl fluorides are resistant to in vivo metabolism. When the imaging moiety comprises a radioactive isotope of fluorine (eg. ¹⁸F), the radiohalogenation may be carried out via direct labelling using the reaction of ¹⁸F-fluoride with a suitable precursor having a good leaving group, such as an alkyl bromide, alkyl mesylate or alkyl tosylate. Alternatively, the radiofluorine atom may be attached via a direct covalent bond to an aromatic ring such as a benzene ring. For such aryl systems, the precursor suitably comprises an activated nitroaryl ring, an aryl diazonium salt, or an aryl trialkylammonium salt. The direct radiofluorination of biomolecules is, however, often detrimental to sensitive functional groups since these nucleophilic reactions are carried out with anhydrous [¹⁸F]fluoride ion in polar aprotic solvents under strong basic conditions. Precursors of Formula (II) when R¹-R³ are C₁₋₈ alkyl also exhibit significant instability under basic conditions. Therefore direct radiofluorination of precursors of the imaging agent of the present invention is not a preferred labelling method. Examples of preferred methods for radiofluorination involves the use of radiolabelled synthons that are conjugated selectively to precursors of the imaging agent of Formula (II), as discussed above for the labelling of radiohalogens in general.

¹⁸F can also be introduced by N-alkylation of amine precursors with alkylating agents such as ¹⁸F(CH₂)₃OMs (where Ms is mesylate) to give N—(CH₂)₃ ¹⁸F, O-alkylation of hydroxyl groups with ¹⁸F(CH₂)₃OMs, ¹⁸F(CH₂)₃OTs or ¹⁸F(CH₂)₃Br or S-alkylation of thiol groups with ¹⁸F(CH₂)₃₀ Ms or 18F(CH₂)₃Br. ¹⁸F can also be introduced by alkylation of N-haloacetyl groups with a ¹⁸F(CH₂)₃OH reactant, to give —NH(CO)CH₂—O—(CH₂)₃ ¹⁸F derivatives or with a ¹⁸F(CH₂)₃SH reactant, to give —NH(CO)CH₂S(CH₂)₃ ¹⁸F derivatives. ¹⁸F can also be introduced by reaction of maleimide-containing precursors with ¹⁸F(CH₂)₃SH. For aryl systems, ¹⁸F-fluoride nucleophilic displacement from an aryl diazonium salt, an aryl nitro compound or an aryl quaternary ammonium salt are suitable routes to aryl-¹⁸F labelled synthons useful for conjugation to precursors of the imaging agent.

Precursors of Formula (II) wherein Y¹ comprises a primary amine group can also be labelled with ¹⁸F by reductive amination using ¹⁸F—C₆H₄—CHO as taught by Kahn et al [J. Lab. Comp. Radiopharm. 45, 1045-1053 (2002)] and Borch et al [J. Am. Chem. Soc. 93, 2897 (1971)]. This approach can also usefully be applied to aryl primary amines, such as compounds comprising phenyl-NH₂ or phenyl-CH₂NH₂ groups.

An especially preferred method for ¹⁸F-labelling of precursors of Formula (II) is when Y¹ comprises an aminooxy group of formula —NH(C═O)CH₂—O—NH₂ which is condensed with ¹⁸F—C₆H₄—CHO under acidic conditions (eg. pH 2 to 4). This method is particularly useful when R¹-R³ of Formulae (I) and (II) are C₁₋₈ alkyl etc. which renders the precursor particularly base-sensitive.

Further details of synthetic routes to ¹⁸F-labelled derivatives are described by Bolton, J. Lab. Comp. Radiopharm., 45, 485-528 (2002). Examples of specific precursors and the associated products are given in Table 3:

TABLE 3
Precursors for ¹⁸F-labelling and the corresponding products

| R^{a} | R^{b} |
|---|---|
| 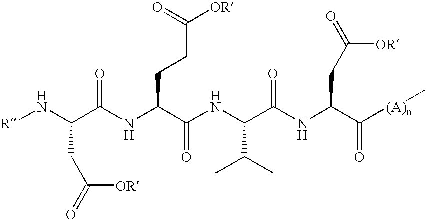 | 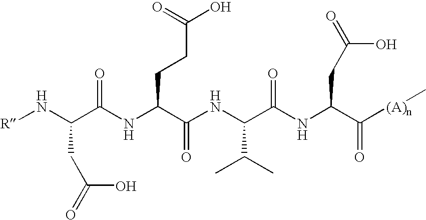 |
| Precursor | Product |
| 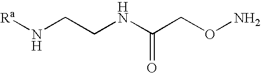 | 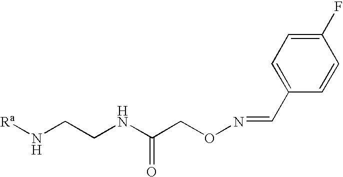 |
| 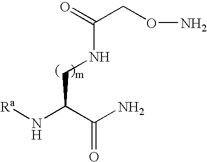 | 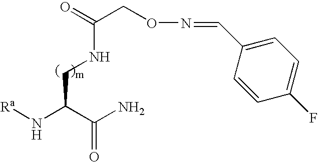 |
| 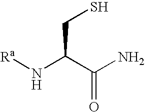 | 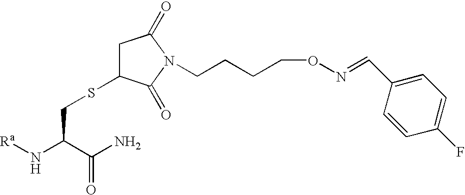 |
| 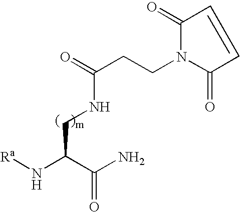 | 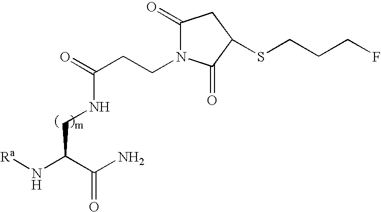 |
| 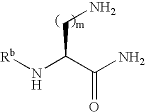 | 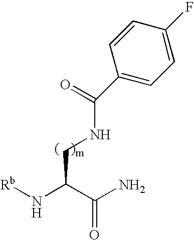 |
| 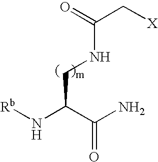 | 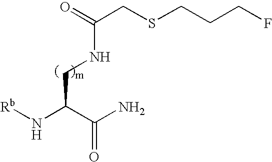 |
| 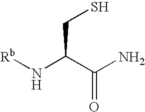 | 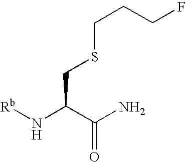 |

R′ is chosen from H, C₁₋₈ alkyl, C₂₋₈ alkoxyalkyl, C₅₋₁₂ aryl or C₅₋₁₆ aralkyl;
R″ = Z¹—(X¹)ₘ₁—
n = 1-10
m = 1-4
y = C₁₋₁₀alkyl, alkylaryl
X = Cl or Br.

In a third aspect, the present invention provides a radiopharmaceutical composition which comprises the imaging agent as described above, together with a biocompatible carrier, in a form suitable for mammalian administration. The “biocompatible carrier” is a fluid, especially a liquid, in which the imaging agent can be suspended or dissolved, such that the composition is physiologically tolerable, ie. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is isotonic); an aqueous solution of one or more tonicity-adjusting substances (eg. salts of plasma cations with biocompatible counterions), sugars (e.g. glucose or sucrose), sugar alcohols (eg. sorbitol or mannitol), glycols (eg. glycerol), or other non-ionic polyol materials (eg. polyethyleneglycols, propylene glycols and the like).

Preferably the biocompatible carrier is pyrogen-free water for injection or isotonic saline.

Such radiopharmaceuticals are suitably supplied in either a container which is provided with a seal which is suitable for single or multiple puncturing with a hypodermic needle (e.g. a crimped-on septum seal closure) whilst maintaining sterile integrity. Such containers may contain single or multiple patient doses. Preferred multiple dose containers comprise a single bulk vial (e.g. of 10 to 30 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose, or “unit dose” and are therefore preferably a disposable or other syringe suitable for clinical use. The pre-filled syringe may optionally be provided with a syringe shield to protect the operator from radioactive dose. Suitable such radiopharmaceutical syringe shields are known in the art and preferably comprise either lead or tungsten.

The radiopharmaceuticals of the present invention may be prepared from kits, as is described in the fourth embodiment below. Alternatively, the radiopharmaceuticals may be prepared under aseptic manufacture conditions to give the desired sterile product. The radiopharmaceuticals may also be prepared under non-sterile conditions, followed by terminal sterilisation using e.g. gamma-irradiation, autoclaving, dry heat or chemical treatment (e.g. with ethylene oxide). Preferably, the radiopharmaceuticals of the present invention are prepared from kits.

In a fourth aspect, the present invention provides kits for the preparation of the radiopharmaceutical compositions of the third embodiment. Such kits comprise the “precursor” of the second embodiment, preferably in sterile non-pyrogenic form, so that reaction with a sterile source of the radioisotope gives the desired radiopharmaceutical with the minimum number of manipulations. Such considerations are particularly important for radiopharmaceuticals where the radioisotope has a relatively short half-life, and for ease of handling and hence reduced radiation dose for the radiopharmacist. Hence, the reaction medium for reconstitution of such kits is preferably a “biocompatible carrier” as defined above, and is most preferably aqueous.

Suitable kit containers comprise a sealed container which permits maintenance of sterile integrity and/or radioactive safety, plus optionally an inert headspace gas (eg. nitrogen or argon), whilst permitting addition and withdrawal of solutions by syringe. A preferred such container is a septum-sealed vial, wherein the gas-tight closure is crimped on with an overseal (typically of aluminium). Such containers have the additional advantage that the closure can withstand vacuum if desired eg. to change the headspace gas or degas solutions.

The non-radioactive kits may optionally further comprise additional components such as a radioprotectant, antimicrobial preservative, pH-adjusting agent or filler.

By the term “radioprotectant” is meant a compound which inhibits degradation reactions, such as redox processes, by trapping highly-reactive free radicals, such as oxygen-containing free radicals arising from the radiolysis of water. The radioprotectants of the present invention are suitably chosen from: ascorbic acid, para-aminobenzoic acid (ie. 4-aminobenzoic acid), gentisic acid (ie. 2,5-dihydroxybenzoic acid) and salts thereof with a biocompatible cation. By the term “biocompatible cation” is meant a positively charged counterion which forms a salt with an ionised, negatively charged group, where said positively charged counterion is also non-toxic and hence suitable for administration to the mammalian body, especially the human body. Examples of suitable biocompatible cations include: the alkali metals sodium or potassium; the alkaline earth metals calcium and magnesium; and the ammonium ion. Preferred biocompatible cations are sodium and potassium, most preferably sodium.

By the term “antimicrobial preservative” is meant an agent which inhibits the growth of potentially harmful micro-organisms such as bacteria, yeasts or moulds. The antimicrobial preservative may also exhibit some bactericidal properties, depending on the dose. The main role of the antimicrobial preservative(s) of the present invention is to inhibit the growth of any such micro-organism in the radiopharmaceutical composition post-reconstitution, ie. in the radioactive diagnostic product itself. The antimicrobial preservative may, however, also optionally be used to inhibit the growth of potentially harmful micro-organisms in one or more components of the non-radioactive kit of the present invention prior to reconstitution. Suitable antimicrobial preservative(s) include: the parabens, ie. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the reconstituted kit is within acceptable limits (approximately pH 4.0 to 10.5) for human or mammalian administration. Suitable such pH-adjusting agents include pharmaceutically acceptable buffers, such as tricine, phosphate or TRIS [ie. tris(hydroxymethyl)aminomethane], and pharmaceutically acceptable bases such as sodium carbonate, sodium bicarbonate or mixtures thereof. When the conjugate is employed in acid salt form, the pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

Preferred aspects of the “precursor” when employed in the kit are as described for the second embodiment above. The precursors for use in the kit may be employed under aseptic manufacture conditions to give the desired sterile, non-pyrogenic material. The precursors may also be employed under non-sterile conditions, followed by terminal sterilisation using e.g. gammna-irradiation, autoclaving, dry heat or chemical treatment (e.g. with ethylene oxide). Preferably, the precursors are employed in sterile, non-pyrogenic form. Most preferably the sterile, non-pyrogenic precursors are employed in the sealed container as described above. The “precursor” of the kit is preferably supplied covalently attached to a solid support matrix as describe for the second embodiment.

In a fifth aspect, the present invention discloses the use of the imaging agent of the first embodiment for the diagnostic imaging in vivo of disease states of the mammalian body where caspase-3 is implicated, wherein said mammal is previously administered with the radiopharmaceutical composition of the third embodiment.

By “previously administered” is meant that the step involving the clinician, wherein the imaging agent is given to the patient eg. intravenous injection, has already been carried out. This embodiment includes the use of the imaging agent of the first embodiment for the manufacture of diagnostic agent for the diagnostic imaging in vivo of disease states of the mammalian body where caspase-3 is implicated.

Such non-invasive imaging would relate to caspase-3 in abnormal apoptosis, and would be useful in monitoring cell death in a number of diseases. It is believed that apoptosis imaging would be valuable in pathologies where cell proliferation and apoptosis are high, eg. myocardial infarction, aggressive tumours and transplant rejection. Such imaging would also be of value in the monitoring of chemotherapeutic drug therapy for these conditions.

In other diseases where apoptosis is thought to be important, but the number of apoptotic events is relatively rare such as in Alzheimer's disease, the available cell pool would be small and hence much more difficult to visualise. It is therefore believed likely that the apoptosis imaging agents of the present invention are best applied to pathologies where apoptosis is relatively acute, such as that seen in myocardial infarctions, aggressive tumours and transplant rejection. For those diseases in which apoptosis is more chronic, such as neuropathologies and less aggressive tumours, there may be insufficient apoptotic cells to register above background.

Essentially all treatments for cancer, including radiotherapy, chemotherapy or immunotherapy, are intended to induce apoptosis in their tumour cell targets. The imaging of apoptosis may have the capability for providing rapid, direct assessment or monitoring of the effectiveness of tumour treatment which may fundamentally alter the way cancer patients are managed. It is anticipated that patients whose tumours are responding to therapy will show significantly increased uptake of the imaging agent due to the elevated apoptotic response in the tumour. Patients whose tumours will not respond to further treatment may be identified by the failure of their tumours to increase uptake of the imaging agent post-treatment.

Excessive apoptosis is associated with a wide range of human diseases, and the importance of caspases in the progression of many of these disorders has been demonstrated. Hence, the imaging agents of the present invention are useful for the in vivo diagnostic imaging and or therapy monitoring in a range of disease states, which include:
(a) acute disorders, such as response to cardiac and cerebral ischaemia/reperfusion injury (eg. myocardial infarction or stroke respectively), spinal cord injury, traumatic brain injury, organ rejection during transplantation, liver degeneration (eg. hepatitis), sepsis and bacterial meningitis;
(b) chronic disorders such as neurodegenerative diseases (eg. Alzheimer's disease, Huntington's Disease, Down's Syndrome, spinal muscular atrophy, multiple sclerosis, Parkinson's disease), immunodeficiency diseases (eg. HIV), arthritis, atherosclerosis and diabetes;

The monitoring of efficacy for agents used to induce apoptosis in cancers such as: bladder, breast, colon, endometrial, head and neck, leukaemia, lung, melanoma, non-Hodgkins lymphoma, ovarian, prostate and rectal.

The evaluation of therapeutic intervention in cancer patients with measurable disease has several applications:
the evaluation of the anti-neoplastic activity of new anti-cancer drugs;
to determine efficacious therapeutic regimens;
the identification of the optimal dose and dosing schedules for new anticancer drugs;
the identification of optimal dose and dosing schedules for existing anticancer drugs and drug combinations;
the more efficient stratification of cancer patients in clinical trials into responders and non-responders of therapeutic regimens;
the efficient and timely evaluation of response of individual patients to established therapeutic anticancer regimens.

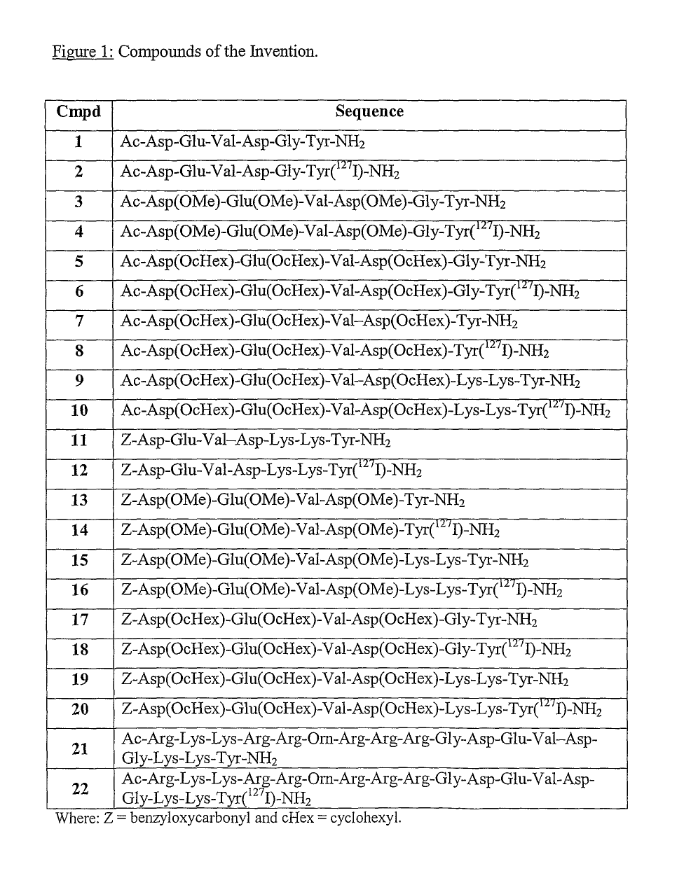

The invention is illustrated by the non-limiting Examples detailed below. Example 1 describes the synthesis of Compounds 1-22 (see FIG. 1). Examples 2 to-8 provide the syntheses of ¹²³I-labelled compounds of the invention (Compounds 2A, 6A, SA, 10A, 14A, 18A and 20A respectively) from suitable precursors. Examples 9 to 11 provide the syntheses of ¹⁸F-labelled compounds suitable for ¹⁸F radiolabelling of caspase-3 substrates of the invention. Example 12 provides in vitro potency data for Compounds 2 and 22. Example 13 provides in vivo plasma stability data for Compounds 2, 6 and 8. Whilst in vivo de-iodination was observed, radiofluorinated or alternative radioiodinated labelling methods as discussed above in the description of the invention would give improved in vivo stability of the radiolabel. These approaches involve substitution of the tyrosine moiety with amino-phenylalanine, histidine or tryptophan for the direct iodination of aniline-, imidazole- or indole-derivatives. Further alternatives involve the use of a synthon approach for conjugation of radioiodinated-phenyl synthons to precursors via a variety of routes. Examples of these compounds are illustrated in Table 2. Radioiodinated phenyl-compounds are likely to give improved in vivo stability since the labelled compound is less susceptible to deiodination than radioiodinated phenolic derivatives such as radioiodinated tyrosine containing peptides.

### EXAMPLE 1

### Synthesis of Compounds 1-22

### a) Peptide Synthesis

The peptidyl resin corresponding to the sequences of Compounds 1-22 in FIG. 1 were assembled by standard solid-phase peptide chemistry [Barany et al, Int. J. Peptide Protein Research 30, 705-739 (1987)] on a Rink Amide resin (from NovaBiochem, typical loading 0.73 mmol/g). An Applied Biosystems (Perkin Elmer) model 433A peptide synthesizer was used. The residues (from the carboxyl terminus) were assembled on a 0.25 mmol scale using single couplings (2.5 hours coupling cycles) of a 4-fold molar excess of Fmoc-amino acids (1 mmol cartridges) pre-activated with 2-(1H-benzotriazol-1-yl)-1,1,3,3-tetramethyluronium-hexafluorophosphate (HBTU)/1-hydroxy-benzotriazole (HOBt)/diisopropylethylamine (DIEA) in NMP. Fmoc-deprotection was achieved with conductivity monitoring using 20% piperidine in N-methylpyrrolidone (NMP). The washing solvent was NMP. The amino acid-side chain protecting groups used were either tert-butyl (tBu) or cyclohexyl (OcHex) for Asp, Glu, tBu for Tyr, Boc for Lys and Orn and (2,2,5,6,7-pentamethyldihyrobenzofuran-5-sulfonyl) (Pbf) for Arg.

Fmoc-¹²⁷I-Tyr-OH was pre-activated with 7-Azabenzotriazol-1-yloxytris (pyrrolidino)-phosphonium-hexafluorophosphate (PyAOP) in dimethylformamide (DMF) containing 4-methylmorpholine (NMM) for ten minutes and then added to the Rink amide resin contained in a manual nitrogen bubbler apparatus [Wellings, D. A., Atherton, E. (1997) in Methods in Enzymology (Fields, G. Ed), 289, p. 53-54, Academic Press, New York]. Further chain elongation was carried out using the peptide synthesizer as described above. After complete assembly of the desired sequences, any undesired acylation of the unprotected hydroxyl group on the ¹²⁷I-iodo-tyrosine side-chain was reversed by treating the peptide resins with 20% piperidine in DMF. Final capping of the N-terminus of the peptide-resins was achieved using either acetic anhydride or benzyl chloroformate in DCM in the presence of NMM.

b) Deprotection and Cleavage from the Resin.

The peptide resins were treated with trifluoroacetic acid (TFA) containing 2.5% triisopropylsilane (TIS) and 2.5% water for 2 hours, using a manual nitrogen bubbler apparatus, in order to cleave the peptides from the resins whilst simultaneously removing all side-chain protecting groups from the peptide, except for OcHex. The cleavage mixtures were filtered and washed with small quantities of neat TFA. The combined filtrate and washings were concentrated by rotary evaporation and then triturated with diethyl ether to obtain the crude peptides. The precipitates were isolated by centrifugation, washed with ether and then lyophilized from 50% ACN-0.1% aq TFA yielding the crude products.

### c) Methylation (Compounds 3, 4, 13, 14, 17, 18)

The crude peptides to be methylated (1 eq, 20 mg) were typically treated with thionyl chloride (20 eq) in methanol (MeOH) (10 mL) at RT. After 60 mL/min the reaction mixtures were concentrated under reduced pressure and the residue lyophilised from 50% ACN-0.1% aq TFA

### d) Purification.

The crude peptides were purified by preparative RP-HPLC. The column (Phenomenex Luna C18 5μ, 22×250 mm) was eluted at 10 mL/min using gradients over 40 min. The eluting buffers were water containing 0.1% TFA and acetonitrile containing 0.1% TFA. The desired peak fractions were pooled and lyophilised affording the pure products.

### d) Characterisation

The peptides were characterised by analytical RP-HPLC and by electrospray MS (Table 3). The C18 column used was either a Phenomenex Luna C18 5μ, 4.6×250 mm column eluted at 1 mL/min or a Phenomenex Luna C18(2) 3μ, 2.0×50 mm column eluted at 0.3 mL/min, using gradients over 20 or 10 min respectively. The eluting buffers were water containing 0.1% TFA (buffer A) and acetonitrile containing 0.1% TFA (buffer B). The eluent was monitored at λ=214 nm and at λ=254 nm.

TABLE 4
Characterisation of Compounds 1-22.

| | | Reten- | | |
|---|---|---|---|---|
| | | tion | MS (M + H⁺) | |
| | | time | m/z | m/z |
| Compound | Gradient | (min) | expected | found |
| 1 | 0-30% B, 10 min | 7.68 | 738.3 | 738.7 |
| 2 | 5-50% B; 20 min | 15.88 | 864.2 | 864.5 |
| 3 | 5-50% B; 10 min | 6.49 | 780.3 | 780.0 |
| 4 | 5-50% B; 10 min | 7.77 | 906.2 | 905.9 |
| 5 | 30-80% B; 10 min | 7.33 | 984.5 | 984.3 |
| 6 | 30-80% B; 10 min | 8.12 | 1110.4 | 1110.3 |
| 7 | 40-80% B; 20 min | 16.98 | 927.5 | 927.8 |
| 8 | 30-80% B; 10 min | 8.36 | 1053.4 | 1053.3 |
| 9 | 20-60% B; 20 min | 16.77 | 1183.7 | 1183.9 |
| 10 | 20-60% B; 20 min | 18.40 | 1309.6 | 1309.9 |
| 11 | 5-50% B, 20 min | 16.90 | 1029.5 | 1029.8 |
| 12 | 10-50% B; 20 min | 14.97 | 1155.4 | 1155.7 |
| 13 | 30-55% B; 20 min | 13.45 | 815.3 | 815.6 |
| 14 | 30-50% B; 20 min | 18.53 | 941.2 | 941.5 |
| 15 | 5-40% B; 10 min | 8.00 | 1071.5 | 1071.5 |
| 16 | 10-60% B; 20 min | 15.90 | 1197.4 | 1197.4 |
| 17 | 40-90% B; 10 min | 7.29 | 1076.6 | 1076.5 |
| 18 | 40-95% B; 20 min | 18.97 | 1202.4 | 1202.6 |
| 19 | 30-60% B; 20 min | 15.18 | 1183.7 | 1183.9 |
| 20 | 20-60% B; 20 min | 18.40 | 1309.6 | 1309.9 |
| 21 | 5-15% B; 20 min | 20.30 | 1179.7 | 1180.0 |
| | | | (MH²⁺) | (MH²⁺) |
| 22 | 10-20% B; 20 min | 15.45 | 1242.6 | 1243.0 |
| | | | (MH²⁺) | (MH²⁺) |

### EXAMPLE 2

### Synthesis of ¹²³I-Labelled Compound 2 (Compound 2A)

### Step (a): ¹²⁷I-Analogue (Compound 2).

Compound 2 was prepared following the reaction scheme:

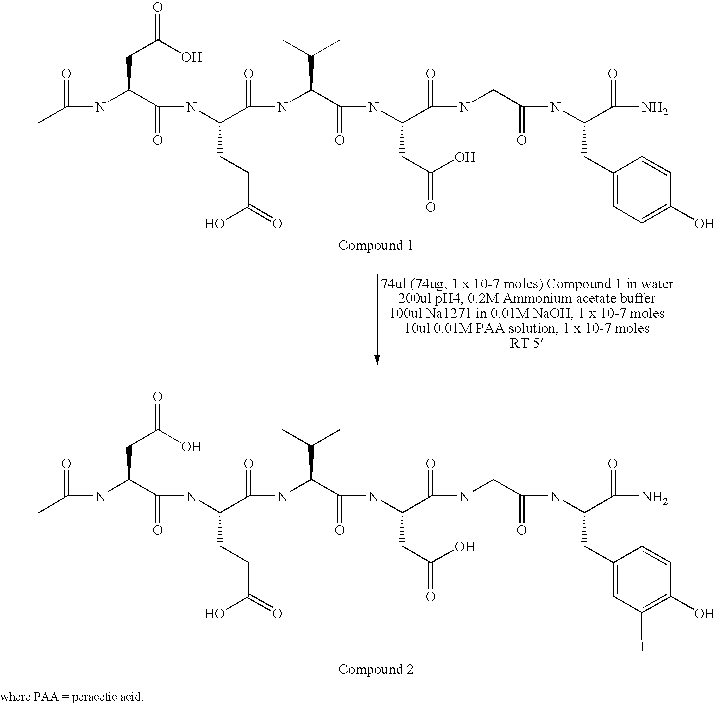

Mass spec analysis of ¹²⁷I prepared and purified material, confirmed identity.

### Step (b): Preparation of Compound 2A.

To Compound 1 (7411g, 1×10⁻⁷ moles) dissolved in 74 μl water, was added 200 μL pH 4, 0.2M ammonium acetate buffer, 10 μl Na¹²⁷I in 0.01MNaOH (1×10⁻⁸ moles), ca. 10-30 μl (150-450 MBq) Na¹²³I in 0.05M NaOH and 10 μl 0.001 M PAA solution (1×10⁻³ moles). [¹²³I]-Compound 2 was HPLC purified and diluted in pH 7.4, 50 mM sodium phosphate buffer to 20 and 100 MBq/ml with specific activities of 14 and 45 MBq/nmole respectively. Co-elution with the 1271 standard was observed confirming identity. Good stability (>90%) was observed over 3.5 hours post dilution.

### EXAMPLE 3

### Synthesis of ¹²³I-Labelled Compound 6 (Compound 6A)

### Step (a): ¹²⁷I-Analogue (Compound 6).

Compound 6 was prepared following the reaction scheme:

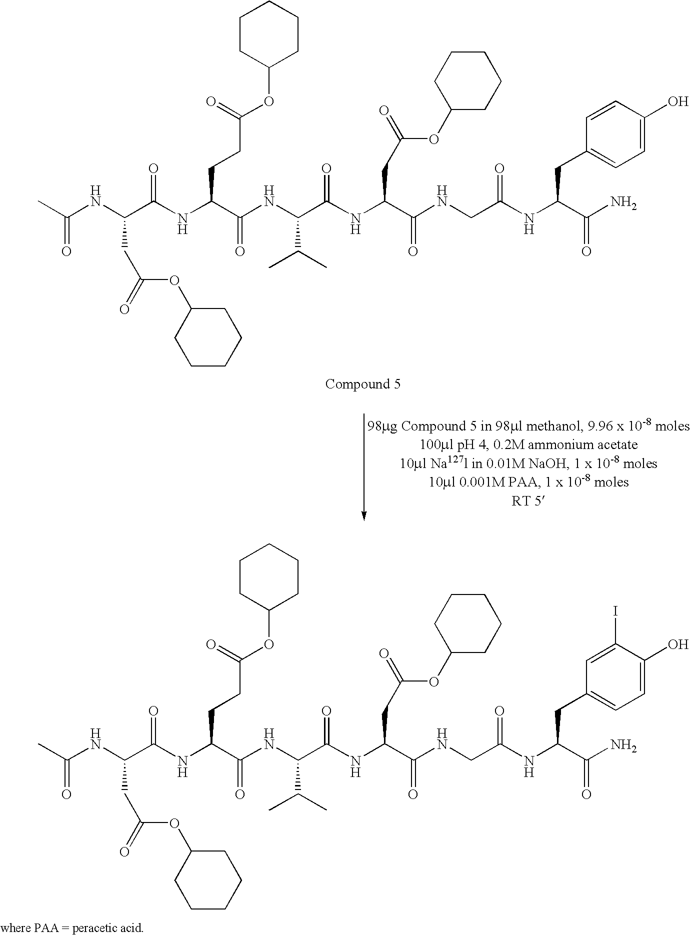

Mass spec analysis of ¹²⁷I prepared and purified material, confirmed identity.

### Step (b): Preparation of Compound 6A.

Compound 5 (98 μg, 1×10⁻⁷ moles) dissolved in 98 μl methanol, was added to 100 μl pH4, 0.2M ammonium acetate buffer, 10 μl Na¹²⁷I in 0.01M NaOH (1×10⁻⁸ moles), ca. 10-30 μl (150-450 MBq) Na¹²³I in 0.05M NaOH and 10 μl 0.001M PAA solution (1×10⁻⁸ moles) post iodonium formation. Compound 6A was HPLC purified and diluted in pH6, 50 mM sodium phosphate buffer to 20 and 100 MBq/ml with specific activities of 13 and 43 MBq/nmole respectively. 10% ethanol was added to aid solubility. Co-elution with the ¹²⁷I standard was observed confirming identity and Compound 6A. Good stability (>⁹⁰%) was observed over 3.5 hours post dilution.

### EXAMPLE 4

### Synthesis of ¹²³I-Labelled Compound 8 (Compound 8A)

### Step (a): ¹²⁷I-Analogue (Compound 8).

Compound 8 was prepared following the reaction scheme:

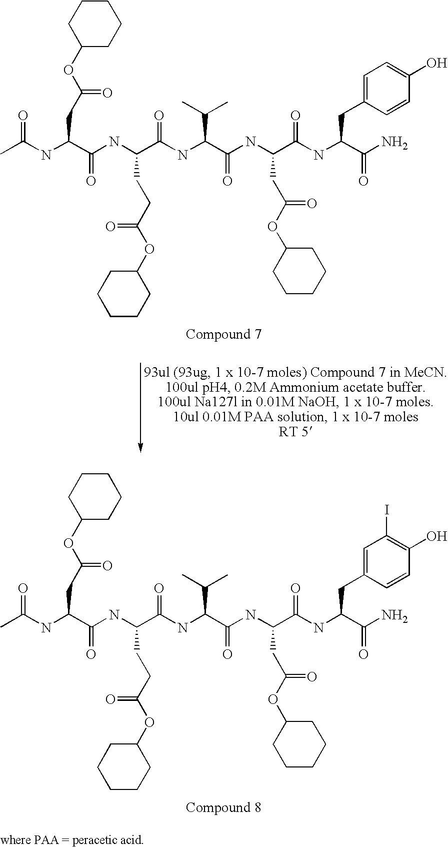

Mass spec analysis of ¹²⁷I prepared and purified material, confirmed identity.

### Step (b): Preparation of Compound 8A.

Compound 7 (93 μg, 1×10⁻⁷ moles) dissolved in 93 μl acetonitrile, was added to 100 μl pH4, 0.2M ammonium acetate buffer, 10 μl Na¹²⁷I in 0.01MNaOH (1×10⁻⁸ moles), ca. 10-30 μl (150-450 MBq) Na¹²³I in 0.05M NaOH and 10 μl 0.001NM PAA solution (1×10⁻⁸ moles) post iodonium formation. [¹²³I]-Compound 8 was HPLC purified and diluted in pH7.4, 50 mM sodium phosphate buffer to 20 and 100 MBq/ml with specific activities of 10 and 40 MBq/nmole respectively. 10% ethanol was added to aid solubility. Co-elution with the ¹²⁷I standard was observed confirming identity. Good stability (>90%) was observed over 4 hours post dilution.

### EXAMPLE 5

### Synthesis of ¹²³I-Labelled Compound 10 (Compound 10A)

### Step (a): ¹²⁷I-Analogue (Compound 10).

Compound 10 was prepared following the reaction scheme:

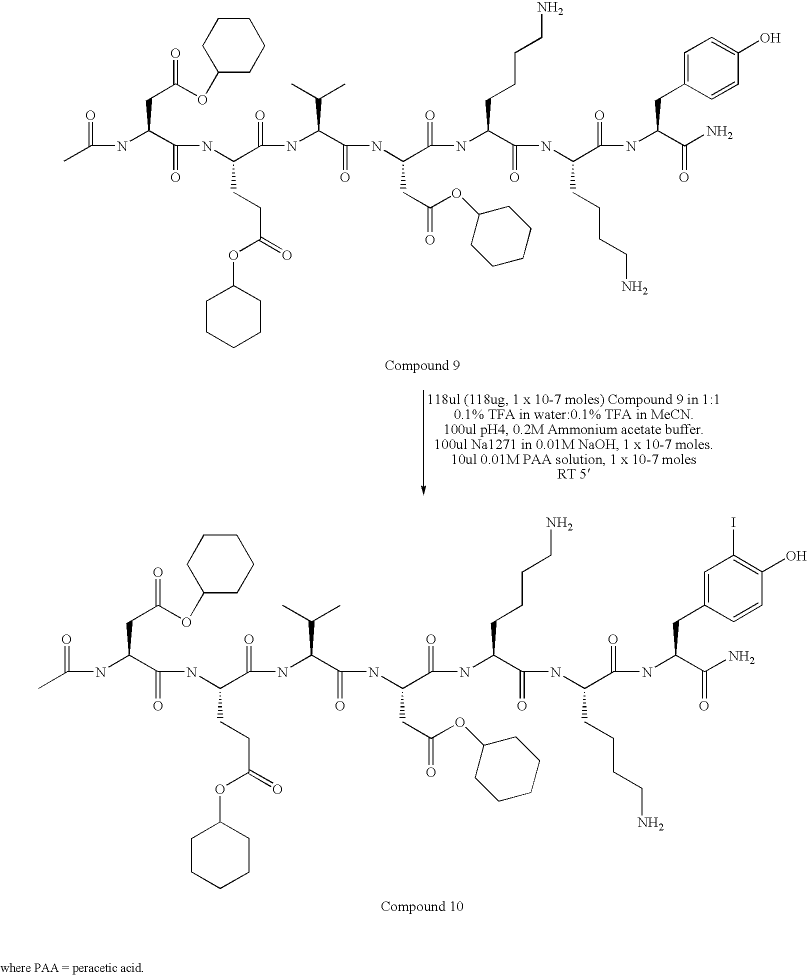

Mass spec analysis of ¹²⁷I prepared and purified material, confirmed identity.

### Step (b): Preparation of Compound 10A.

Compound 9(118 μg, 1×10⁻⁷ moles) dissolved in 118 μl 1:1 0.1% TFA in water: 0.1% TFA in acetonitrile, was added to 2001 pH4, 0.2M ammonium acetate buffer, 10 μl Na¹²⁷I in 0.01M NaOH (1×10⁻⁸ moles), ca. 10-30 μl (150-450 MBq) Na¹²³I in 0.05M NaOH and 10 μl MPAA solution (1×10⁻⁸ moles) post iodonium formation. [¹²³I]-Compound 10 was HPLC purified and diluted in pH 7.4, 50 mM sodium phosphate buffer to 20 and 100 MBq/ml with specific activities of 12 and 401Bq/nmole respectively. 10% ethanol was added to aid solubility. Co-elution with the ¹²⁷I standard was observed confirming identity. Good stability (>85%) was observed over 4 hours post dilution.

### EXAMPLE 6

### Synthesis of ¹²³I-Labelled Compound 14 (Compound 14A)

### Step (a): ¹²⁷I-Analogue (Compound 14).

Compound 14 was prepared following the reaction scheme below:

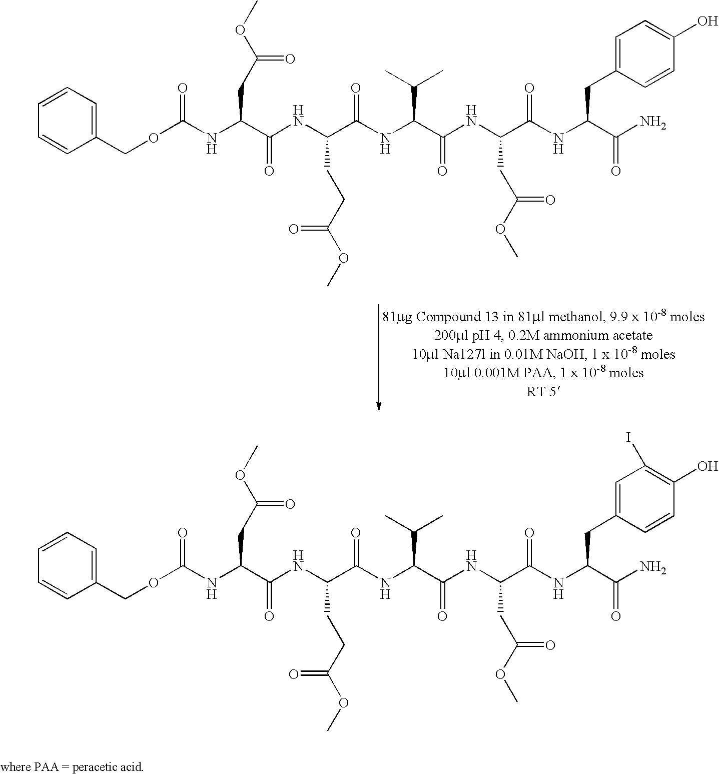

Mass spec analysis of ¹²⁷I prepared and purified material, confirmed identity.

### Step (b): Preparation of Compound 14A.

Compound 13 (81 μg, 1×10⁻⁷ moles) upon dissolution in 81 μl methanol, was added to 200 μl 0.2M, pH4 ammonium acetate buffer, 10 μl Na¹²⁷I in 0.01M NaOH (1×10⁻⁸ moles), ca. 30 μl (450 MBq) Na¹²³I in 0.05M NaOH and 10 μl 0.001M PAA solution (1×10⁻⁸ moles) post iodonium formation. Compound 14A was HPLC purified and diluted in pH6, 50 mM sodium phosphate buffer to 100 MBq/ml with a specific activity of 41 MBq/nmole. Co-elution with the 1271 standard was observed confirming identity and Compound 14A. Good stability (>90%) was observed over 3.5 hours post dilution.

### EXAMPLE 7

### Synthesis of ¹²³I-Labelled Compound 18 (Compound 18A)

### Step (a): ¹²⁷I-Analogue (Compound 18).

Compound 18 was prepared following the reaction scheme below.

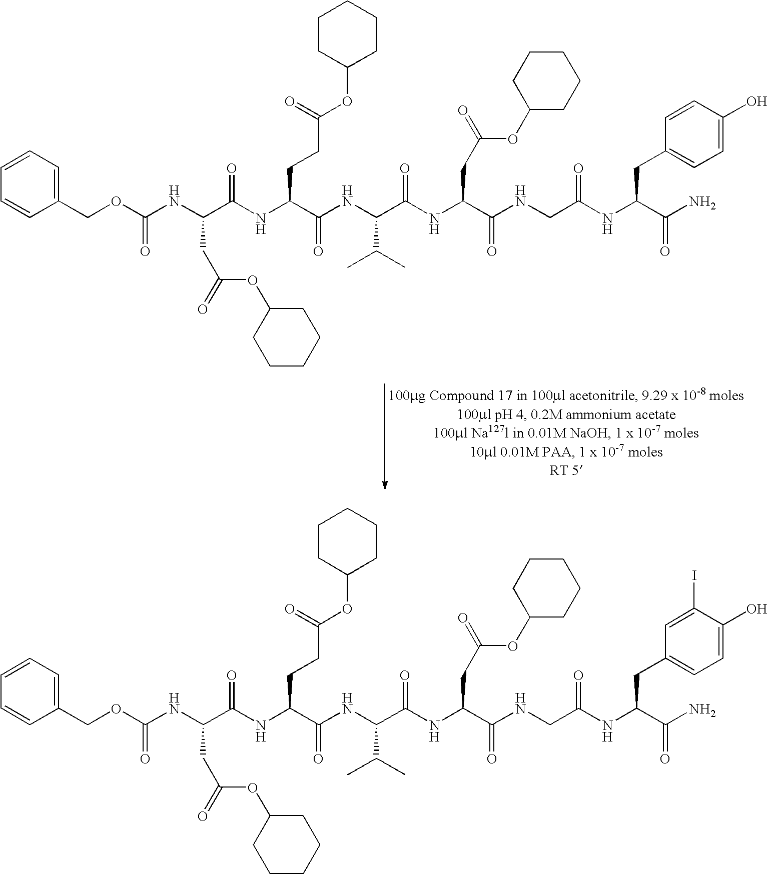

Mass spec analysis of ¹²⁷I prepared and purified material, confirmed identity.

### Step (b): Preparation of Compound 18A.

Compound 17 (100 μg, 9.29×10⁻⁸ moles) upon dissolution in 100 μl acetonitrile, was added to 100 μl 0.2M, pH4 ammonium acetate buffer, 10 μl Na¹²⁷I (1×10⁻⁸ moles), ca. 10-30 μl Na¹²³I in 0.05M NaOH (150-450 MBq) and 10 μl 0.001M PAA solution (1×10⁻⁸ moles) post iodonium formation. Compound 18A was HPLC purified and diluted in pH7.4, 50 mM sodium phosphate buffer to 20 and 100 MBq/ml with typical specific activities of 10 and 40 MBq/nmole respectively. 10% ethanol was added to aid solubility. Co-elution with the ¹²⁷I standard was observed confirming identity and compound 18A. Good stability (>90%) was observed over 3.5 hours post dilution.

### EXAMPLE 8

### Synthesis of ¹²³1-Labelled Compound 20 (Compound 20A)

Step (a): 1271- analogue (Compound 20).

Compound 20 was prepared following the reaction scheme below.

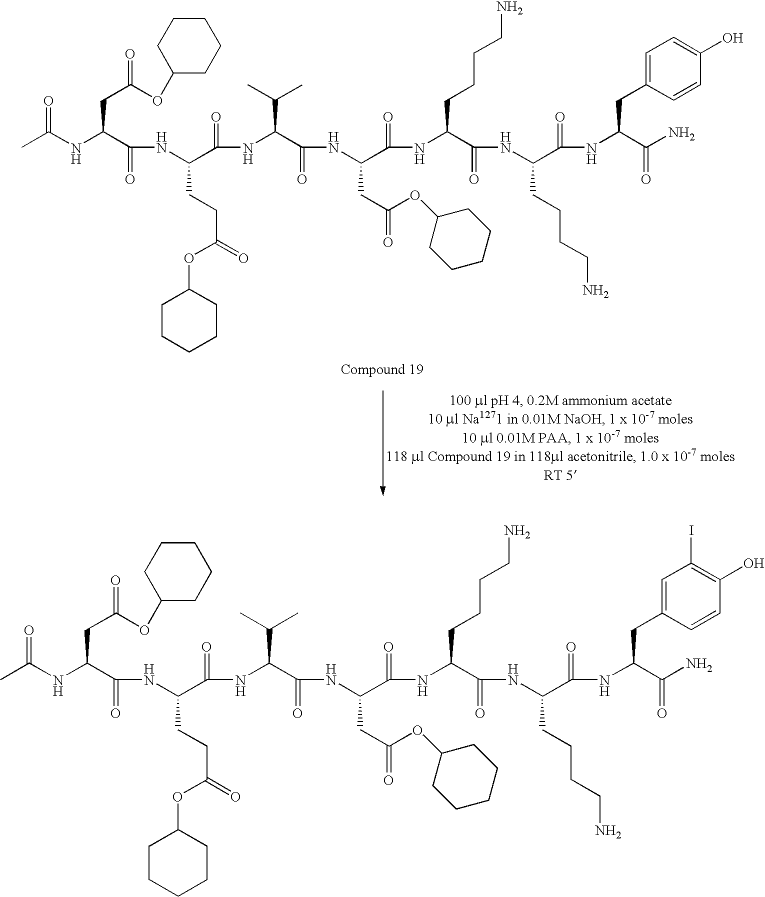

Both Compound 20 and the corresponding di-iodinated species were purified and their identities confirmed by Mass spec analysis.

### Step (b): Preparation of Compound 20A.

Compound 19 (118 μg, 1×10⁻⁷ moles) upon dissolution in 118 μl 1:1 0.1% TFA in water: 0.1% TFA in acetonitrile, was added to 200 μl 0.2M, pH4 ammonium acetate buffer, 10 μl Na¹²⁷I in 0.01M NaOH (1×10⁻⁸ moles), ca. 10-30 μl (150-450 MBq) Na¹²³I in 0.05MNaOH and 10 μl 0.001M PAA solution (1×10⁻⁸ moles) post iodonium formation. Compound 20A was HPLC purified and diluted in pH7.4, 50 mM sodium phosphate buffer to 20 and 100 MBq/ml with specific activities of typically 13 and 41 MBq/nmole respectively. Co-elution with the ¹²⁷I standard was observed confirming identity and Compound 20A. Good stability (>90%) was observed over 3.5 hours post dilution.

### EXAMPLE 9

### Synthesis of the ¹⁸F-Labelled Derivative for N-Alkylation

Synthesis of 3-[¹⁸F]fluoropropyl tosylate

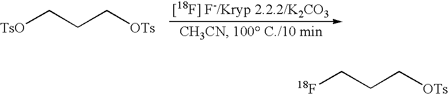

Via a two-way tap Kryptofix 222 (10 mg) in acetonitrile (300 μL) and potassium carbonate (4 mg) in water (300 μL), prepared in a glass vial, was transferred using a plastic syringe (1 ml) into a carbon glass reaction vessel sited in a brass heater. ¹⁸F-fluoride (185-370 MBq) in the target water (0.5-2 ml) was then added through the two-way tap. The heater was set at 125° C. and the timer started. After 15 mins three aliquots of acetonitrile (0.5 ml) were added at 1 min intervals. The ¹⁸F-fluoride was dried up to 40 mins in total. After 40 mins, the heater was cooled down with compressed air, the pot lid was removed and 1,3-propanediol-di-p-tosylate (5-12 mg) and acetonitrile (1 ml) was added. The pot lid was replaced and the lines capped off with stoppers. The heater was set at 100° C. and labelled at 100° C./10 mins. After labelling, 3-[¹⁸F]fluoropropyl tosylate was isolated by Gilson RP HPLC using the following conditions:

| | |
|---|---|
| Column | u-bondapak C18 7.8 × 300 mm |
| Eluent | Water (pump A): Acetonitrile (pump B) |
| Loop Size | 1 ml |
| Pump speed | 4 ml/min |
| Wavelength | 254 nm |
| Gradient | 5-90% eluent B over 20 min |
| Product Rt | 12 min |

Once isolated, the cut sample (ca. 10 ml) was diluted with water (10 ml) and loaded onto a conditioned C18 sep pak. The sep pak was dried with nitrogen for 15 mins and flushed off with an organic solvent, pyridine (2 ml), acetonitrile (2 ml) or DMF (2 ml). Approx. 99% of the activity was flushed off.

3-[¹⁸F]fluoropropyl tosylate is used to N-alkylate amines by refluxing in pyridine.

### EXAMPLE 10

### [¹⁸F]-Thiol Derivative for S-Alkylation

Step (a): Preparation of 3-[¹⁸F]fluoro-tritylsulfanyl-propane.

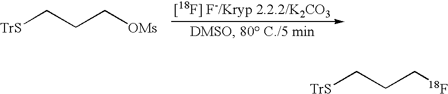

Via a two-way tap Kryptofix 222 (10 mg) in acetonitrile (800 μL) and potassium carbonate (1 mg) in water (50 μL), prepared in a glass vial, was transferred using a plastic syringe (1 ml) to the carbon glass reaction vessel situated in the brass heater. ¹⁸F-fluoride (185-370 MBq) in the target water (0.5-2 ml) was then also added through the two-way tap. The heater was set at 125° C. and the timer started. After 15 mins three aliquots of acetonitrile (0.5 ml) were added at 1 min intervals. The ¹⁸F-fluoride was dried up to 40 mins in total. After 40 mins, the heater was cooled down with compressed air, the pot lid was removed and trimethyl-(3-tritylsulfanyl-propoxy)silane (1-2 mg) and DMSO (0.2 ml) was added. The pot lid was replaced and the lines capped off with stoppers. The heater was set at 80° C. and labelled at 80° C./5 mins. After labelling, the reaction mixture was analysed by RP HPLC using the following HPLC conditions:

| Column | u-bondapak C18 7.8 × 300 mm | |
|---|---|---|
| Eluent | 0.1% TFA/Water (pump A): 0.1% | |
| | TFA/Acetonitrile (pump B) | |
| Loop Size | 100 ul | |
| Pump speed | 4 ml/min | |
| Wavelength | 254 nm | |
| Gradient | 1 mins | 40% B |
| | 15 mins | 40-80% B |
| | 5 mins | 80% B |

The reaction mixture was diluted with DMSO/water (1:1 v/v, 0.15 ml) and loaded onto a conditioned t-C18 sep-pak. The cartridge was washed with water (10 ml), dried with nitrogen and 3-[¹⁸F]fluoro-1-tritylsulfanyl-propane was eluted with 4 aliquots of acetonitrile (0.5 ml per aliquot).

Step (b): Preparation of 3-[¹⁸F]fluoro-propane-1-thiol

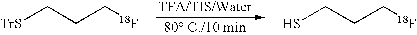

A solution of 3-[¹⁸F]fluoro-1-tritylsulfanyl-propane in acetonitrile (1-2 ml) was evaporated to dryness using a stream of nitrogen at 100° C./10 mins. A mixture of TFA (0.05 ml), triisopropylsilane (0.01 ml) and water (0.01 ml) was added followed by heating at 80° C./10 mins to produce 3-[¹⁸F]fluoro-propane-1-thiol.

Step (c): Reaction with —N(CO)CH₂Cl Precursors.

A general procedure for labelling a chloroacetyl precursor is to cool the reaction vessel containing the 3-[¹⁸F]fluoro-1-mercapto-propane from Step (b) with compressed air, and to then to add ammonia (27% in water, 0.1 ml) and the precursor (1 mg) in water (0.05 ml). The mixture is heated at 80° C./10 mins.

### EXAMPLE 11

### Synthesis of ¹⁸F-Labelled Derivatives Via Benzaldehyde

### Step (a): 4-¹⁸F-benzaldehyde.

To a flat-bottom carbon glass reaction vessel (4 ml) was added Kryptofix 222 (5 mg) in acetonitrile (800 μl) and potassium carbonate [13.5 mg/ml (H₂O), ca. 0.1M] (50 μl) were added. The vessel was placed in a brass heater and the reaction vessel lid fitted with 3 PTFE lines was tightened down. Line 1 was fitted with a 2-way tap, line 2 was connected to a waste vial and line 3 was blanked off. The experimental set-up was placed behind a lead wall. ¹⁸F-Fluoride contained in the cyclotron target water (370-740 MBq; 0.5-2 ml) was added through the two-way tap. The N₂ line was connected to the 2-way tap and the heater was set at 110° C. At 10 min after heating was started, the N₂ line was removed and an aliquot of acetonitrile (0.5 ml) was added. This process was repeated at ca. 10.5 and 11 min after heating was started. Following each addition of acetonitrile the N₂ line was reconnected to the 2-way tap. A second nitrogen line was connected to the capped off line 3, to flush out any liquid present in this line. The ¹⁸F-Fluoride was dried up to 30 mins in total.

After 30 mins, the heater was cooled down with compressed air, the reaction vessel lid was removed and 4-(trimethylammonium)benzaldehyde trifluoromethane sulfonate [prepared by the method of Poethko et al, J. Nucl. Med., 45(5) p 892-902 (2004); 0.5-0.8 mg, 0.0016-0.0026 mmol] in DMSO (1000 μl) was added. The 3 PTFE lines were capped off with stoppers. The reaction vessel was heated at 90° C./15 min to yield 4-¹⁸F-benzaldehyde (typical incorporation yield ca.50%). The crude product was used without further purification.

### Step (b): Conjugation Procedure.

The primary amine-functionalised precursor (0.003 mmol) is dissolved in citric acid/Na₂HPO₄ buffer [500 μl; which can be prepared by mixing 809 μL of a 0.1M aqueous citric acid solution with 110 μL of a 0.2M aqueous solution of anhydrous Na₂HPO₄], and then added directly to 4-¹⁸F-benzaldehyde (crude) from Step (a). The reaction vessel is then heated at 70° C./15 mins to yield the crude product.

### Step (c): Work-Up Procedure and Formulation.

The whole reaction mixture from step (b) is diluted with water to a volume of ca. 20 ml and loaded onto conditioned t-C18 sep pak [conditioned with DMSO (5 ml) followed by water (10 ml)]. The loaded t-C18 sep was subsequently flushed with water (2×5 ml) followed with DMSO (3×5 ml). The combined DMSO flushes, containing the desired products, were purified using RP HPLC preparative system:

| | |
|---|---|
| Column | LunaC18(2) 10 × 100 mm (5 u) |
| Eluent | Water (pump A): Acetonitrile (pump B) |
| Loop Size | 2 ml |
| Flow rate | 3 ml/min |
| Wavelength | 254 nm |

The separated HPLC peak was diluted with water to a volume of ca. 20 ml and loaded onto a conditioned t-C18 sep pak [conditioned with ethanol (5 ml) followed with water (10 ml)]. The loaded t-C18 sep pak was subsequently flushed with water (1×5 ml) followed with ethanol (3×0.2 ml, 1×0.4 ml). The combined ethanol flush, containing the desired products, was evaporated to a volume of ca. 0.1 ml and formulated to ca. 10% ethanol with phosphate-buffered saline (PBS, 1 ml). pH of formulated compounds was ca. 7.

### EXAMPLE 12

### In Vitro Potency of Compounds 2 and 22

Potency (Ki) of the caspase-3 test substrates was evaluated using a commercially available caspase-3 assay kit (BIOMOL QuantiZyme™ Assay System, CASPASE-3 Assay Kit for Drug Discovery). A summary of the data generated is shown in Table 5. The DEVD substrate sequence was bio-modified with the additional of both tyrosine residues for radiolabelling and leader sequence to aid cell penetration. In vitro potency was maintained following both of these bio-modifications.

TABLE 5
In vitro potency data

| COMPOUND | MEAN KI (μM) |
|---|---|
| Ac-Asp-Glu-Val-Asp-AMC | 7.74 |
| (Calbiochem Cat # 235425) | |
| Compound 2 | 7.83 |
| Compound 22 | 9.24 |

### EXAMPLE 13

### In Vivo Plasma Stability of Compounds 2A, 6A and 8A

In vivo stability studies were carried out with ¹²³I-Compound 2, ¹²³I-Compound 6 and ¹²³I-Compound 8 (Compounds 2A, 6A and 8A respectively). Radiolabelled compounds were injected intravenously into mate Wistar rats (ca. 200-300 g). Blood samples were collected at several time points post injection, spun down to plasma and analysed by HPLC. Comparison of the HPLC profiles we made with control samples (compounds spiked into plasma in vitro) and used to assess the degree of metabolism in vivo.

In vivo stability studies of ¹²³I-Compound 6 (Compound 6A) showed compound instability and de-iodination over time with the formation of an array of metabolites. Metabolite retention times correlated with tie free acid form, protecting groups and iodo-tyrosine. In vivo stability study of ¹²³I-Compound 8 (Compound 8A) was carried out to determine whether the absence of asp-glycine in the compound would lead to improved in vivo stability. Results showed rapid compound denaturation and formation of metabolites and de-iodination. Fewer metabolites were produced compared to Compound 6A. In vivo stability study of ¹²³I-Compound 2 (Compound 2A) was carried out to ascertain whether the formation of metabolites with Compounds 6A and 8A was the result of cyclohexyl protecting group cleavage. However, Compound 2A also demonstrated compound instability in vivo over time with the formation of an array of metabolites and de-iodination.

What is claimed is:

1. An imaging agent which comprises a labelled caspase-3 substrate of Formula I:
Z¹-(X¹)ₘ₁-Asp(R¹)-Xaa1-Xaa2-Asp(R²)-(A)ₙ-[IM] (I)
where:
Z¹ is attached to the N-terminus of X¹ or the Asp residue, and is H or a metabolism inhibiting group;
X¹ is a cell membrane permeable leader sequence peptide of 4 to 20 amino acids which facilitates cell membrane transport from the outside to the inside of a mammalian cell in vivo;
Xaa1 is Glu(R³) or Met;
Xaa2 is Val or is Gln when Xaa1 is Met;
Asp is aspartic acid;
-(A)ₙ- is a linker group wherein each A is independently —CR₂—, —CR═CR—, —C≡C—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group, an amino acid, a sugar or a monodisperse polyethyleneglycol (PEG) building block;
each R is independently chosen from H, C₁₋₄ alkyl, C₂₋₄ alkenyl, C₂₋₄ alkynyl, C₁₋₄ alkoxyalkyl or C₁₋₄ hydroxyalkyl;
R¹, R² and R³ are independently R′ groups which are attached at the carboxy side chain of the Asp or Glu amino acid residue, where each R′ is chosen from H, C₁₋₈ alkyl, C₂₋₈ alkoxyalkyl, C₅₋₁₂ aryl or C₅₋₁₆ aralkyl;
m₁ is 0 or 1;
n is an integer of value 0 to 10;
IM is an imaging moiety which comprises a gamma-emitting radioactive halogen or a positron-emitting radioactive non-metal, wherein following administration of said labelled caspase-3 substrate to the mammalian body in vivo, the imaging moiety can be detected externally in a non-invasive manner.

2. The imaging agent of claim 1, wherein when m₁ is 0, at least one of R¹, R² and R³ is C₁₋₈ alkyl.

3. The imaging agent of claim 1, wherein when m₁ is 1, at least one of R¹, R² and R³ is H.

4. The imaging agent of claim 1, where each R is independently chosen from methyl and cyclohexyl.

5. The imaging agent of claim 1, where Z¹ is acetyl or benzyloxycarbonyl.

6. The imaging agent of claim 1, where (A)ₙ is (Gly)ₙ or (Lys)ₙ.

7. The imaging agent of claim 1, where X¹ comprises a leader sequence chosen from: KWSFRVSYRGISYRRSR, AWSFRVSYRGISYRRSR, RKKRRQRRR, RRLSYSRRRF, RGGRLSYSRRRFSVSVGR, RGGRLSYSRRRFSTSTGR, RKKRR-Orn-RRR, RRRRRRRRR and β-(VRR)₄, where Orn is ornithine.

8. The imaging agent of claim 1, where the gamma-emitting radioactive halogen is ¹²³I.

9. The imaging agent of claim 1, where the positron-emitting radioactive non-metal is chosen from ¹⁸F, ¹¹C, ¹²⁴I or ¹³N.

10. A precursor suitable for the preparation of the imaging agent of claim 1, which comprises a compound of Formula II:
Z¹-(X¹)ₘ₁-Asp(R¹)-Xaa1-Xaa2-Asp(R²)-(A)ₙ-[Y¹] (II)
where Z¹, X¹, m₁, R¹, Xaa1, Xaa2, Asp, R², A and n are as defined in claim 1, and Y¹ is a non-radioactive group which comprises a substituent capable of reaction with a source of the positron-emitting radioactive non-metal or gamma-emitting radioactive halogen to give the imaging agent of Formula I.

11. The precursor of claim 10, wherein the substituent of the Y¹ group is chosen from:
(i) an organometallic derivative such as a trialkylstannane or a trialkylsilane;
(ii) a derivative containing an alkyl halide, alkyl tosylate or alkyl mesylate for nucleophilic substitution;
(iii) a derivative containing an aromatic ring activated towards nucleophilic or electrophilic substitution;
(iv) a derivative containing a functional group which undergoes facile alkylation;
(v) a derivative which alkylates thiol-containing compounds to give a thioether-containing product;
(vi) a derivative which undergoes condensation with an aldehyde or ketone;
(vii) a derivative which is acylated by an active ester group.

12. The precursor of claim 10 which is in sterile, apyrogenic form.

13. The precursor of claim 10, where the precursor is bound to a solid phase.

14. The precursor of claim 10, where the source of the positron-emitting radioactive non-metal or gamma-emitting radioactive halogen is chosen from:
(i) halide ion or F⁺ or I⁺; or
(ii) an alkylating agent chosen from an alkyl or fluoroalkyl halide, tosylate, triflate or mesylate.

15. A radiopharmaceutical composition which comprises the imaging agent of claim 1 together with a biocompatible carrier, in a form suitable for mammalian administration.

16. The radiopharmaceutical composition of claim 15, which has a radioactive dose suitable for a single patient and is provided in a suitable syringe or container.

17. A kit for the preparation of the radiopharmaceutical composition which comprises the imaging agent of claim 1 together with a biocompatible carrier, in a form suitable for mammalian administration, which further comprises the precursor suitable for the preparation of the imaging agent of claim 1 which comprises a compound of Formula II:
Z¹-(X¹)ₘ₁-Asp(R¹)-Xaa1-Xaa2-Asp(R²)-(A)ₙ-[Y¹] (II)

18. where Z¹, X¹, m₁, R¹, Xaa1, Xaa2, Asp, R², A and n are as defined in claim 1, and Y¹ is a non-radioactive group which comprises a substituent capable of reaction with a source of the positron-emitting radioactive non-metal or gamma-emitting radioactive halogen to give the imaging agent of Formula I.

19. The kit of claim 17 where the precursor is in sterile, apyrogenic form.

20. Use of the imaging agent of claim 1 in a method of diagnosis of a caspase-3 implicated disease state of the mammalian body, wherein said mammal is previously administered with the radiopharmaceutical compositions which comprise the imaging agent of claim 1 together with a biocompatible carrier, in a form suitable for mammalian administration.
